SEQUENCE LISTING (2 sequences)

SEQ ID NO: 1 - DNA, 48 bp, Artificial Sequence
  Description of Artificial Sequence Synthetic construct
gcgccttgtttcgttcttgcgctctccgagggccattgcatggagccg

SEQ ID NO: 2 - DNA, 48 bp, Artificial Sequence
  Description of Artificial Sequence Synthetic construct
cggctccatgcaatggccctcggagagcgcaagaacgaaacaaggcgc

(19) United States
(12) Patent Application Publication
(10) Pub. No.: US 2002/0076751 A1
(43) Pub. Date: Jun. 20, 2002

(54) Novel plasmid, bearing the plasmid, and method of producing an enzyme using the transformant

(76) Inventors: Shizuo Hattori, Tsuruga-shi (JP); Seiji Takeshima, Tsuruga-shi (JP); Atsushi Sogabe, Tsuruga-shi (JP); Yoshihisa Kawamura, Tsuruga-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 09/765,865

(22) Filed: Jan. 18, 2001

(30) Foreign Application Priority Data
Jan. 21, 2000 (JP) 2000-13264

(51) Int. Cl. C12P 21/02; C12N 1/21; C12N 15/74

(52) U.S. Cl. 435/69.1; 435/252.34; 435/320.1

(57) ABSTRACT

The invention relates to a plasmid characterized in that the plasmid comprises a DNA fragment containing a gene coding for an enzyme taking PQQ as the prosthetic group as cloned in a broad-host-range vector defected of conjugative transfer function beforehand and that the plasmid is capable of being expressed in bacteria of the genus Pseudomonas.

### TECHNICAL FIELD

The present invention relates to a recombinant DNA comprising a gene coding for an enzyme taking PQQ (pyrroloquinoline-quinone) as a prosthetic group, a bacterial transformant bearing the same and having an ability to produce said enzyme taking PQQ as a prosthetic group, and a method of producing said enzyme taking PQQ as a prosthetic group which comprises using the transformant.

Enzymes taking PQQ as a prosthetic group, such as glucose dehydrogenase (also designated herein as GDH), may irreversibly reduce artificial electron acceptors and, as such, are suitable for the high-sensitivity assay of various compounds corresponding to the respective enzymes. For example, the GDH mentioned above is suitable for the high-sensitivity assay of glucose.

### BACKGROUND ART

While PQQ was chemically characterized in 1979 as a third coenzyme for dehydrogenases, its presence has so far been confirmed in many forms of life, chiefly with dehydrogenases represented by methanol dehydrogenase in methanol-utilizing bacteria and alcohol dehydrogenase and glucose dehydrogenase in acetic acid bacteria.

These dehydrogenases are capable of reducing artificial electron acceptors and, therefore, by utilizing a dye such as Nitro Blue Tetrazolium, the reactions can be detected in visible light with high sensitivity. Moreover, since the reactions involved are one-direction reactions in contrast to the equilibrium reactions of NAD-dependent dehydrogenases, these are considered to be very useful for the quantitative analysis of trace substances (Methods Enzymol. 89, 20 (1982)).

The most useful of the enzymes taking PQQ as a prosthetic group is PQQ-dependent GDH, which can be used for the determination of blood glucose. Actually, this enzyme can be applied broadly to the color reactions of membrane-immobilized dry reagents and as chip-mounted sensors, for instance, not to speak of its use as a routine biochemical reagent. Compared with glucose oxidase and NAD(P)-dependent GDH, the substrate of which is also glucose, this enzyme is characterized in that it is not susceptible to dissolved oxygen and that, because of the simplicity of reaction, devices can be constructed easily and at low cost.

Cloning of the above PQQ-dependent GDH gene has been reported in Acinetobacter calcoaceticus LMD79.41 (J. Bacteriol., 170, 2121 (1988) and Mol. Gen. Genet., 217, 430 (1989)), Escherichia coli (J. Bacteriol., 172, 6308 (1990)) and Gluconobacter suboxydans (Mol. Gen. Genet., 229, 206 (1991)), among others, and its expression in E. coli has also been reported.

It is known that enterobacteria represented by E. coli which has been well analyzed genetically and optimized as the host for transformation do not produce PQQ and that even if E. coli is transformed with a vector harboring a gene fragment coding for a PQQ-dependent GDH and cultured, only the apoenzyme GDH which is inactive can be obtained. Such apo-GDHs may be converted to active holo-GDHs by supplying PQQ exogenously but the necessary PQQ is a very expensive reagent. Moreover, it has been confirmed that, in large-scale production, the apoenzyme is not totally converted to the holoenzyme.

Biotechnol. Lett., 16, 12, 1265 (1994) reports on the production of an active holo-GDH by using a medium supplemented with PQQ in the culture of a transformant E. coli but this technique also requires a large amount of the expensive PQQ. It is also reported in Mol. Gen. Genet., 229, 206 (1991) that the objective gene product could be expressed by inserting a gene fragment encoding the GDH of Gluconobacter oxydans into its own chromosome but this technique is disadvantageous in that because the gene is located on the chromosome and its copy number is limited, the amount of expression is small.

The inventors of the present invention found previously, as disclosed in JP Kokai H11-243949, that GDH can be produced on a large scale at low cost by growing a strain of microorganism provided with an ability to produce a PQQ-dependent GDH by transformation with a vector harboring a DNA fragment containing the gene coding for the PQQ-dependent GDH. As an example, there was described a method of producing GDH which comprises using a transformant Pseudomonas putida as transformed with the expression vector pGLD3 constructed by subcloning a GDH-encoding gene into the broad-host-range vector pTS1137.

The expression vector pTS1137 used in the above production system is based on R1b679 which is structurally identical to RSF1010 and R1162 belonging to the incompatibility group P-4, with the introduction of a promoter (pTN8 promoter) which is suspected to be derived from TOL plasmid. Since the above promoter is capable of constitutive expression, a high production of the objective gene can be expected in the absence of an inducer. However, like R1b679, pTS1137 does not bear the self-transfer gene tra but bears the conjugative-transfer gene mob, thus having the risk for conjugation to other microbial strains. Thus, should a recombinant strain bearing the above plasmid leak out in commercial-scale production and contact a microbial strain bearing a helper plasmid like RP4, it would happen that the GDH-containing plasmid is transferred, thus posing a safety problem with containment of the transformant.

### BRIEF DESCRIPTION OF THE DRAWINGS

FIG. 1 is a diagram illustrating the scheme used for constructing pTM33 from pTS1137.

### BRIEF SUMMARY OF INVENTION

The inventors of the present invention conducted an extensive research toward the above end and found that an enzyme taking PQQ as the prosthetic group can be produced safely at a high expression level by using a broad-host-range vector deprived of its conjugative transfer function. The present invention has accordingly been developed.

The present invention, therefore, has the following constitution.

(1) A plasmid characterized in that the plasmid comprises a DNA fragment containing a gene coding for an enzyme taking PQQ as the prosthetic group as cloned in a broad-host-range vector defected of conjugative transfer function beforehand and that the plasmid is capable of being expressed in bacteria of the genus Pseudomonas.

(2) The plasmid according to (1) wherein the broad-host-range vector is a plasmid belonging to the incompatibility group P-4.

(3) The plasmid according to (1) wherein the enzyme taking PQQ as the prosthetic group is glucose dehydrogenase.

(4) A transformant characterized in that the transformant comprises the plasmid according to (1) as introduced into a bacterial strain capable of producing an enzyme taking PQQ as the prosthetic group.

(5) The transformant according to (4) wherein the strain capable of producing an enzyme taking PQQ as the prosthetic group is a bacterial strain of the genus Pseudomonas.

(6) A method of producing an enzyme taking PQQ as the prosthetic group characterized in that the method comprises growing the transformant according to (4) in a nutrient medium to let it produce the enzyme taking PQQ as the prosthetic group in the culture broth and harvesting the enzyme taking PQQ as the prosthetic group from said culture broth.

### DETAILED DESCRIPTION OF INVENTION

In the present invention, a DNA fragment containing a gene coding for an enzyme taking PQQ as the prosthetic group can be harvested from a microorganism capable of producing the particular enzyme. By way of illustration, a gene fragment containing a GDH gene can be harvested from Acinetobacter calcoaceticus, Escherichia coli or Gluconobacter suboxydans. A gene fragment containing an alcohol dehydrogenase gene can be harvested from Pseudomonas putida, Pseudomonas aeruginosa or Pseudomonas testosteroni, for instance, a gene fragment containing a glycerol dehydrogenase gene can be harvested from Pseudomonas putida, for instance, and a gene fragment containing a sorbitol dehydrogenase gene can be harvested from Gluconobacter suboxydans, for instance.

Those genes coding for enzymes taking PQQ as the prosthetic group may be extracted from such bacterial strains or synthesized chemically. These may also be obtained by using the PCR technique.

In the practice of the present invention, the following techniques, inter alia, can be used for acquiring a gene coding for an enzyme taking PQQ as the prosthetic group.

Acquisition of a GDH gene coding for the enzyme taking PQQ as the prosthetic group from Acinetobacter calcoaceticus NCIMB11517, for instance, can be carried out as follows. After isolation and purification of the chromosome of Acinetobacter calcoaceticus NCIMB11517, the DNA is fragmented by sonication and restriction enzyme cleavage and each of these fragments is ligated to a linear expression vector at the blunt end or cohesive end of the DNAs with a DNA ligase to construct a circular recombinant vector. The recombinant vector is transferred to a host microorganism capable of replicating the particular vector and a screening is made using the vector's marker and the expression of the enzyme activity as indicators to select clones bearing the recombinant vector harboring the gene coding for PQQ-dependent GDH.

Then, the strain bearing said recombinant vector is cultured and the recombinant vector is separated and purified from the cultured cells. Then, the gene coding for GDH can be isolated from the expression vector.

The gene donor strain is cultured under agitation for 1 to 3 days, the resulting culture is centrifuged, and the cell pellet is lysed to prepare a PQQ-dependent GDH gene-containing lysate. Said lysis can be effected by means of a lytic enzyme such as lysozyme, optionally in combination with a protease or other enzyme or a surfactant such as sodium dodecyl sulfate (SDS). Furthermore, physical disruption means such as freeze-thaw treatment or French pressure cell lysis can be used in combination.

To separate and purify DNA from such a lysate, the conventional deproteinization procedure, such as phenol or protease treatment, ribonuclease treatment, alcohol precipitation and other techniques can be used in a suitable combination.

The technology of fragmenting the DNA isolated and purified from the microorganism includes sonication and restriction enzyme digestion, among other methods. Preferably, the use of a type II restriction enzyme acting on a defined nucleotide sequence is suited.

As the vector for cloning, a vector constructed for gene recombination from a phage or plasmid capable of autonomous replication in the host microorganism is suitable. As to the phage, Lambda gt10 or Lambda gt11 may for example be used when the host microorganism is Escherichia coli. The plasmid may for example be pBR322, pUC19 or pBluescript when E. coil is used as the host microorganism.

In cloning, a vector fragment can be acquired by digesting the above vector with the same restriction enzyme as used for fragmenting the DNA from the GDH-encoding gene donor but it is not essential to use the same restriction enzyme as used in the fragmentation of the microbial DNA. For example, after the cohesive end of the bacterial DNA fragment is annealed to the cohesive end of the vector fragment, a recombinant vector consisting of the bacterial DNA fragment and vector DNA fragment is constructed using a suitable DNA ligase. Where necessary, the annealing product may be transferred into a host microorganism to construct the desired vector by taking advantage of the endogenous DNA ligase of the host.

The host microorganism for use in the above cloning is not particularly restricted insofar as the recombinant vector to be used may be stable therein and capable of autonomous replication for transduction of the foreign gene. Generally, Escherichia coli W3110, E. coli C600, E. coli HB101, E. coli JM109 and E. coli DH5α, among others, can be employed.

The technology of introducing a recombinant vector into a host microorganism, taking E. coli as an example of the host, includes but is not limited to the calcium-treated competent cell method and the electroporation method.

The transformant thus obtained can be cultured in a nutrient medium to produce the enzyme taking PQQ as the prosthetic group stably in a large amount. The test for successful introduction of the desired vector into the host bacterial cell can be made by screening for clones expressing both the drug-resistant marker of the vector harboring the objective DNA and the enzyme activity on addition of PQQ. For example, the strain of microorganism which grows in a selection medium based on the drug-resistant marker and produces the PQQ-dependent enzyme can be selected.

The transfer from a recombinant vector harboring the PQQ-dependent GDH-encoding gene so generally selected to a recombinant vector which can be replicated in a microorganism capable of producing the PQQ-dependent GDH can be easily carried out by recovering the enzyme gene DNA from the recombinant vector harboring the PQQ-dependent enzyme gene by restriction enzyme digestion or PCR and ligating the same to another vector fragment. Furthermore, the transformation of a microorganism capable of producing PQQ-dependent GDH with such a vector can be carried out by, for example, the calcium-treated competent cell method or the electroporation method.

As the microorganism capable of producing the PQQ-dependent GDH enzyme, there can be mentioned methanol-utilizing bacteria, such as those belonging to the genus Methylobacterium, acetic acid bacteria of the genus Acetobacter or the genus Gluconobacter, and bacteria belonging to the genus Flavobacterium, the genus Pseudomonas or the genus Acinetobacter. Among these, bacteria of the genera Pseudomonas and Acinetobacter are preferred because the usable host-vector systems have been well established, thus making them easy to use.

Among bacteria of the genus Pseudomonas, P. aeruginosa, P. fluorescens, P. putida, etc. can be employed. Among bacteria of the genus Acinetobacter, A. calcoaceticus, A. baumanni, etc. can be employed.

As the vector that can be replicated in the above microorganism, vectors derived from Pseudomonas species can be mentioned. These have been classified as the incompatibility groups P-1 through P-11 (Idenshi Kumikae Jitsuyoka Gijutsu (Practical Recombinant DNA Technology), Vol. 4, pp. 73-85, published by Silence Forum (1983)).

As specific examples, there can be mentioned RP4, RP1 and RK2 in Group P-1; pMG1 in Group P-2; pMG7 in Group P-3; RSF1010, R1b679 and R1162 in Group P-4; Rms163 in Group P-5; Rms149 in Group P-6; Rsm148 in Group P-7; FP2 in Group P-8; R2 in Group P-9; pSR in Group P-10; RP1-1 and pSa in Group P-11. Among these, the plasmids in Groups P-1, P-3, P-4 and P-6, for instance, are known as broad-host-range vectors. These broad-host-range vectors can be used as vectors capable of replication in various gram-negative bacteria and some gram-positive bacteria.

Among the above-mentioned plasmids, all in Groups P-1 through P-11 but those in Group P-4 have the self-transmissible gene tra and are not amenable to biological containment. Therefore, it is preferable to use a plasmid from Group P-4. However, Thomas et al. in J. Bacteriol., 141, 213 (1980), reported on the construction of a miniplasmid pRK290 from the plasmid RK2 of Group P-1 and since this plasmid is deficient in self-transmissible function, similar attempts may be made with a broad range of plasmids inclusive of those belonging to the other groups.

However, the plasmids RSF1010, R1b679 and R1162 of said Group P-4 and the high-expression vectors pTS1137 and pRK290 belonging to the same incompatibility group have mob gene exhibiting a conjugative transfer function on conjugation with Group P-1 plasmids and are not amenable to a perfect biological containment.

As the plasmid ligated to a broad-host-range vector deprived of conjugative transfer function in advance and capable of expression in bacteria of the genus Pseudomonas, there can be mentioned pTM33 derived from said high-expression vector pTS137. Also preferred are RSF1010-derived pED401, pED403, pED405, pED407, pED409, pED412, pED359, pED141, pED142, pED144, pED145 and pED146 which are described in Mol. Gen. Genet., 206, 161 (1987) and the RSF1010-derived RSF1010Δ18, RSF1010Δ13, RSF1010Δ20 and RSF1010Δ201 which are described in Gene, 113, 101 (1992).

Culture of the transformant host can be carried out under conditions judiciously selected with reference to the nutritional physiologic characters of the host and, in many instances, is carried out by the liquid culture technique. On an industrial scale, aeration-agitation culture is preferred.

As nutrient sources of the culture medium, those in routine use for the culture of microorganisms in general can be liberally employed. The carbon source may be any carbonaceous substance that can be assimilated, thus including glucose, sucrose, lactose, maltose, molasses and pyruvic acid, among others. The nitrogen source may be any nitrogenous compound that can be utilized by the microorganism, thus including peptone, meat extract, yeast extract, casein hydrolyzate, and soybean cake alkali-extract, among others. Aside from the above nutrients, phosphates, carbonates, sulfates, salts of magnesium, calcium, potassium, iron, manganese, zinc, etc., certain amino acids, certain vitamins, etc. are used as necessary.

The cultural temperature may vary within the range allowing growth of the microorganism and production of the PQQ-dependent enzyme but is preferably about 20 to about 42° C. in ordinary cases. The cultural time varies somewhat depending on other conditions but the incubation may be discontinued at an appropriate time when the yield of the PQQ-dependent enzyme becomes maximal. Generally, the cultural time is about 12 to about 72 hours. The pH of the medium can be judiciously adjusted within the range allowing growth of the microorganism and production of the PQQ-dependent enzyme but is preferably about 6.0 to about 9.0.

The culture fluid containing the PQQ-dependent enzyme-producer cells can be recovered and used as such. Generally, however, when the PQQ-dependent enzyme occurs in the liquid phase of the culture, the enzyme-containing fraction is separated from the microbial cells by filtration or centrifugation, for instance, and then used. When the enzyme occurs intracellularly, the cells are harvested from the culture fluid by a suitable means such as filtration or centrifugation and disrupted by a mechanical means or enzymatically with, for example, lysozyme, optionally followed by solubilization with a chelating agent, such as EDTA, and a surfactant to provide the enzyme in the form of an aqueous solution.

The PQQ-dependent enzyme-containing solution thus obtained can then be concentrated under reduced pressure or using a membrane and subjected to salting-out with ammonium sulfate, sodium sulfate or the like or fractional precipitation using a hydrophilic organic solvent such as methanol, ethanol, acetone or the like. Heat-treatment and isoelectric focusing are also effective purification means. Then, adsorbent treatment, gel filtration, adsorption chromatography, ion exchange chromatography, affinity chromatography or the like can be carried out to provide a pure enzyme.

For example, a pure enzyme can be obtained by a purification procedure such as gel filtration using Sephadex Gel (Pharmacia Biotech) or the like or column chromatography on DEAE-Sepharose CL-6B (Pharmacia Biotech), octyl-Sepharose CL-6B (Pharmacia Biotech) or the like. This pure enzyme preferably has been purified to the degree giving a single band on an electropheretogram (SDS-PAGE).

The pure enzyme obtained as above can be powdered by lyophilization, vacuum drying or spray drying for distribution. The purified enzyme in this form can be dissolved in phosphate buffer, Tris-HCl buffer or Good's buffer and used. Furthermore, this enzyme taking PQQ as the prosthetic group can be further stabilized by adding calcium ion or a salt thereof, amino acids such as glutamic acid, glutamine, lysine, etc., and, further, serum albumin or the like.

In the present invention, the assay of PQQ-dependent GDH activity is performed under the following conditions.

<Reagents>

50 mM PIPES buffer (pH 6.5)

0.2 mM PMS

0.2 mM NTB

30.6 mM Glucose

0.19% Triton X-100

<Assay conditions>

The mixture of the above reagents, 3 ml, is preliminarily incubated at 37° C. for about 5 minutes and, then, 0.1 ml of the enzyme solution is added. After gentle stirring, the absorbance at 570 nm is recorded over 5 minutes with a spectrophotometer controlled at 37° C. using water as blank and the change in absorbance per minute is read from the linear segment of the curve. As a blank experiment, distilled water in lieu of the enzyme solution was added to the reagent mixture and the absorbance was similarly measured. The amount of the enzyme giving 1/2 μmol of diformazan per minute under the above conditions is taken as 1 unit (U).

### Best Mode for Carrying out the Invention

The following examples are further illustrative of the invention.

### EXAMPLE 1

Construction of a Vector Deprived of Conjugative Transfer Function

The broad-host-range vector pTS1137 fragment containing the conjugative transfer gene group mob as cleaved with BamHI and EcoRI was ligated to pBluescript SK(−) as cleaved with the same restriction enzymes to give pTSEB.

Then, mutation primers having the nucleotide sequences shown under SEQ ID NO:1 (sense strand) and SEQ ID NO:2 (antisense strand) were constructed.

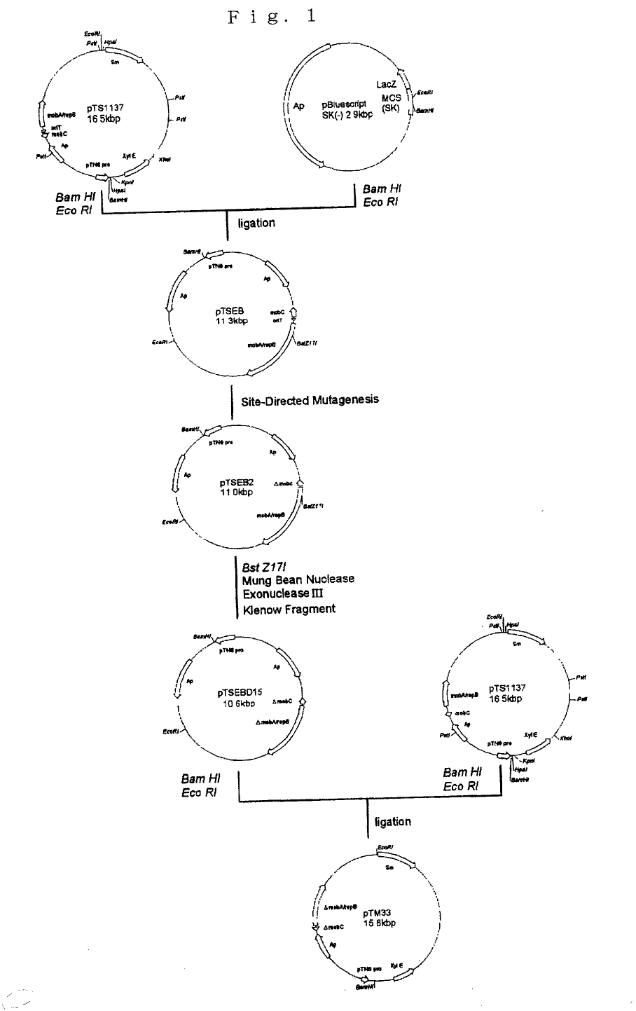

Using the above primers and Transformer Site-Directed Mutagenesis Kit (CLONTECH), a 258 bp fragment containing mobC and oriT was eliminated to give pTSEB2. This pTSEB2 was further cleaved with BstZ17I, digested with Mung Bean Nuclease, Exonuclease III, and repaired with Klenow fragment to give pTSEB15. The pTSEB15 was cleaved with BamHI and EcoRI and ligated to pTS1137 as similarly cleaved with BamHI and EcoRI to give pTM33 (FIG. 1).

### EXAMPLE 2

Evaluation of Conjugative Transfer Function

The conjugative transfer function of the vector constructed in Example 1 was evaluated by the following method.

The pTM33-transformed Escherichia coli JM109, E. coli C600 (RK2) and E. coli XL2-Blue MRF′ were inoculated into LB liquid medium containing 100 μg/ml of streptomycin, LB liquid medium containing 10 μg/ml of kanamycin, and LB liquid medium containing 20 μg/ml of chloramphenicol and shake-cultured at 30° C., 37° C. and 37° C., respectively, until the OD660 value had reached 1 to 2.

The above cultures, 1 ml each, were admixed and the cells were collected on a sterilized nitrocellulose filter (pore size 0.45 μm). This filter was placed on antibiotic-free LB agar and incubated at 33° C. for 4 hours. Then, the cells on the filter were diluted with 2 ml of sterilized saline, streaked on LB agar containing chloramphenicol (Cam; 20 μg/ml) and LB agar containing chloramphenicol (20 μg/ml) and streptomycin (Sm; 100 μg/ml), respectively, and cultured at 33° C. for 24 hours. The grown colonies were then counted. The results are shown in Table 1.

TABLE 1

| Dilution | 10⁻⁰ | 10⁻¹ | 10⁻² | 10⁻³ | 10⁻⁴ | 10⁻⁵ | 10⁻⁶ |
|---|---|---|---|---|---|---|---|
| LB agar medium | — | — | — | — | — | 275 | 28 |
| (Cam & Sm) | | | | | | | |
| LB agar medium | 2 | 0 | — | — | — | — | — |
| (Cam) | | | | | | | |

—: not tested

From the above results, the conjugative transfer efficiency of pTM33 was calculated to be 2×100/275×10⁵=7.3×10⁻⁶. This figure is considerably lower than 2.5×10⁻², the figure similarly found for pTS1137, and indicates that pTM33 has substantially no conjugated transfer function.

### EXAMPLE 3

Construction of an Expression Vector for a Glucose Dehydrogenase Taking PQQ as the Prosthetic Group

From the vector pGLD5 harboring a PQQ-dependent glucose dehydrogenase gene derived from Acinetobacter calcoaseticus NCIMB11517, the GDH gene fragment was excised with BamHI and XhoI and ligated between the BamHI and XhoI sites of the pTM33 constructed in Example 1 to give pGLD6.

### EXAMPLE 4

Construction of a Transformant from a PQQ-producing Microorganism

Pseudomonas putida TN1126 was cultured in LBG medium (LB medium +0.3% glycerol) at 30° C. for 16 hours, at the end of which time the microbial cells were harvested by centrifugation (12000 rpm×10 min) and suspended in 8 ml of ice-cold 300 mM sucrose-5 mM K-phosphate buffer (pH 7.0). The suspension was recentrifuged (12000 rpm×10 min) and the pellet was resuspended in 0.4 ml of ice-cold 300 mM sucrose-5 mM K-phosphate buffer (pH 7.0).

To this suspension was added 0.5 μg of the plasmid DNA (pGLD6) obtained in Example 3, and a transformation was performed by electroporation. From the colonies grown on LB agar medium containing 50 μg/ml of streptomycin, clones having soluble PQQ-dependent GDH activity were isolated.

### EXAMPLE 5

Production of a Glucose Dehydrogenase Taking PQQ as the Prosthetic Group

The transformant obtained in Example 4 was cultured in 50 ml of Terrific broth (1.2% polypeptone, 2.4% yeast extract, 0.4% NaCl, 17 mM KH₂PO₄, 72 MM K₂HP04, pH 7.0) at 30° C. for 24 hours and the resulting culture was centrifuged (12,000 rpm×3 min) to harvest the bacterial cells. The cells were suspended in 50 mM PIPES-NaOH buffer (pH 7.5) containing 1 mM CaCl₂ and disrupted by sonification. The cell debris was removed by centrifugation (12000 rpm×5 min). A crude enzyme solution was then prepared and its GDH activity was assayed. The result is shown in Table 2.

TABLE 2

| Transformant | Expressed activity (U/ml) |
|---|---|
| P. putida TN1126/pGLD6 | 27 |

Enzyme activity per ml of the culture

As described above, by using the plasmid of the invention and growing a transformant bearing the vector defected of conjugative transfer function in advance, the enzyme taking PQQ as the prosthetic group can be produced safely and efficiently at low cost.

1. A plasmid characterized in that the plasmid comprises a DNA fragment containing a gene coding for an enzyme taking PQQ as the prosthetic group as cloned in a broad-host-range vector defected of conjugative transfer function beforehand and that the plasmid is capable of being expressed in bacteria of the genus Pseudomonas.

2. The plasmid according to claim 1 wherein the broad-host-range vector is a plasmid belonging to the incompatibility group P-4.

3. The plasmid according to claim 1 wherein the enzyme taking PQQ as the prosthetic group is glucose dehydrogenase.

4. A transformant characterized in that the transformant comprises the plasmid according to claim 1 as introduced into a bacterial strain capable of producing an enzyme taking PQQ as the prosthetic group.

5. The transformant according to claim 4 wherein the strain capable of producing an enzyme taking PQQ as the prosthetic group is a bacterial strain of the genus Pseudomonas.

6. A method of producing an enzyme taking PQQ as the prosthetic group characterized by its comprising growing the transformant according to claim 4 in a nutrient medium to let it produce the enzyme taking PQQ as the prosthetic group in the culture broth and harvesting the enzyme taking PQQ as the prosthetic group from said culture broth.
